(19) United States
(12) Patent Application Publication
(10) Pub. No.: US 2017/0095610 A1
(43) Pub. Date: Apr. 6, 2017

(54) PERSONAL INJECTION DEVICE

(71) Applicant: MEDTRONIC MINIMED, INC., Northridge, CA (US)

(72) Inventors: Alexander E. Holmes, La Canada, CA (US); Darren Y.K. Yap, Valencia, CA (US)

(73) Assignee: MEDTRONIC MINIMED, INC.

(21) Appl. No.: 14/876,518

(22) Filed: Oct. 6, 2015

(51) Int. Cl. A61M 5/142; A61M 39/24; A61M 5/178

(52) U.S. Cl. CPC A61M 5/14244; A61M 5/178; A61M 5/14212; A61M 39/24

(57) ABSTRACT

A personal injection device wearable by a user to deliver fluid from a reservoir to the user, the personal injection device including: a quartz oscillator operable to generate a constant frequency signal; a control circuit operably connected to the quartz oscillator, the control circuit being operable to generate a drive signal in response to the constant frequency signal; a stator operably connected to the control circuit and defining a rotor gap, the stator being operable to generate an oscillatory field in the rotor gap in response to the drive signal; a magnetic rotor disposed in the rotor gap, the magnetic rotor being operable to rotate in response to the oscillatory field; and a pump operably connected to the magnetic rotor, the pump being operable to move the fluid from the reservoir to the user in response to the rotation of the magnetic rotor.

### TECHNICAL FIELD

The technical field of this disclosure is personal medical systems, particularly, personal injection devices.

### BACKGROUND OF THE INVENTION

Certain medical conditions or diseases require that patients intermittently inject a drug or therapeutic agent subcutaneously to maintain the medical condition or disease under control. Multiple daily injections (MDIs) may be required. One such medical condition is diabetes, for which insulin is injected to regulate blood glucose. An estimated twenty-six million people in the United States, or about 8% of the population, have diabetes. This percentage is expected to increase in the near-term as the population ages.

Insulin pump therapy uses an insulin pump to deliver insulin slowly and continuously throughout the day as basal injections. Unfortunately, delivering fluid continuously and accurately over long periods of time currently requires a complex device to perform and control the pumping. To operate, such complex devices typically employ a programmed microcontroller, which requires significant infrastructure in the form of memory, software, code, power management, and the like. This increases the cost and size of the insulin pump. Many patients are unwilling or unable to use such complex insulin pumps due to the expense, complication, and obtrusiveness.

It would be desirable to have a personal injection device that would overcome the above disadvantages.

### SUMMARY OF THE INVENTION

One aspect of the invention provides a personal injection device wearable by a user to deliver fluid from a reservoir to the user, the personal injection device including: a quartz oscillator operable to generate a constant frequency signal; a control circuit operably connected to the quartz oscillator, the control circuit being operable to generate a drive signal in response to the constant frequency signal; a stator operably connected to the control circuit and defining a rotor gap, the stator being operable to generate an oscillatory field in the rotor gap in response to the drive signal; a magnetic rotor disposed in the rotor gap, the magnetic rotor being operable to rotate in response to the oscillatory field; and a pump operably connected to the magnetic rotor, the pump being operable to move the fluid from the reservoir to the user in response to the rotation of the magnetic rotor.

The foregoing and other features and advantages of the invention will become further apparent from the following detailed description of the presently preferred embodiments, read in conjunction with the accompanying drawings. The detailed description and drawings are merely illustrative of the invention, rather than limiting the scope of the invention being defined by the appended claims and equivalents thereof.

### BRIEF DESCRIPTION OF THE DRAWINGS

FIG. 1 is a block diagram of a personal injection device made in accordance with the invention.

FIGS. 2A & 2B are perspective and top view diagrams, respectively, of a syringe pump for use with a personal injection device made in accordance with the invention.

FIGS. 3A & 3B are schematic diagrams of a cam pump for use with a personal injection device made in accordance with the invention.

FIGS. 4A & 4B are schematic and detail diagrams, respectively, of a rotary pump for use with a personal injection device made in accordance with the invention.

Like elements share like reference numbers in the various drawings.

### DETAILED DESCRIPTION

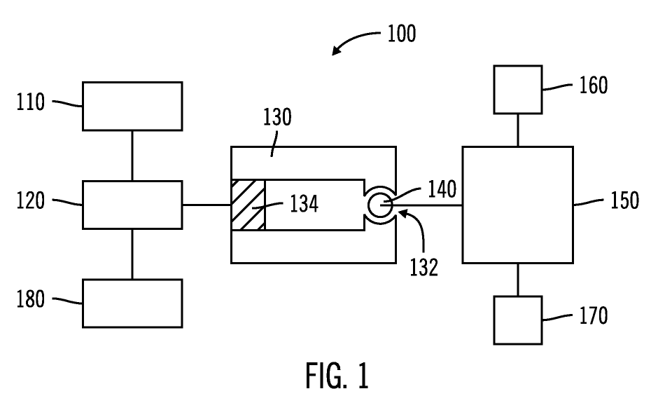

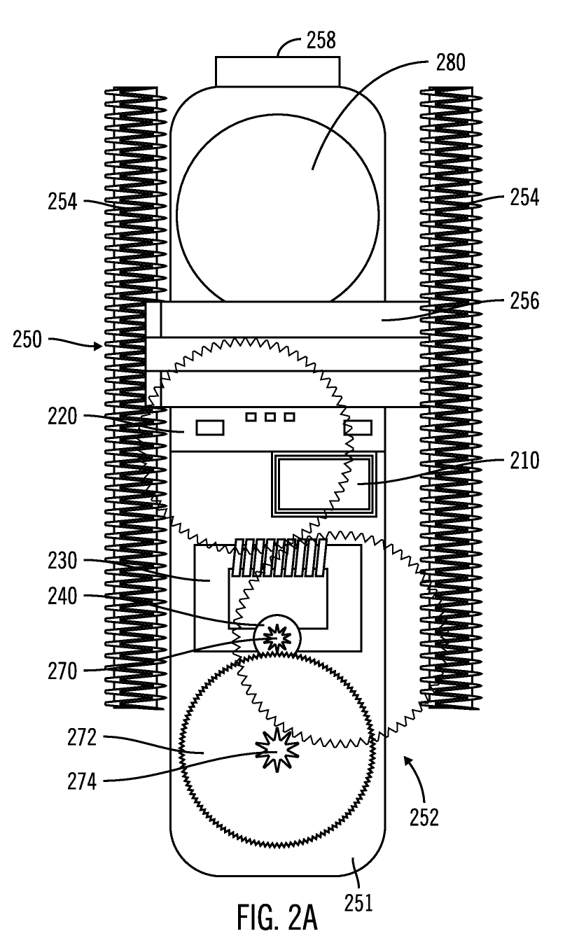

FIG. 1 is a block diagram of a personal injection device made in accordance with the invention. The personal injection device is wearable by a user to deliver fluid from a reservoir to the user. A quartz oscillator provides timing for the personal injection device.

The personal injection device 100 includes a quartz oscillator 110, a control circuit 120 operably connected to the quartz oscillator 110, a stator 130 operably connected to the control circuit 120 and defining a rotor gap 132, a magnetic rotor 140 disposed in the rotor gap 132, and a pump 150 operably connected to the magnetic rotor 140. The pump 150 receives fluid from a reservoir 160 and provides the fluid to a user 170. A battery 180 operably connected to the control circuit 120 provides power for the personal injection device 100. In one example, the battery 180 is a button cell battery.

The quartz oscillator 110 generates a constant frequency signal, which is provided to the control circuit 120, which generates a drive signal in response to the constant frequency signal. The quartz oscillator 110 can receive an excite signal from the control circuit 120 to drive the quartz oscillator 110 at its natural frequency. The stator 130 receives the drive signal and generates an oscillatory field in the rotor gap 132 in response. A coil 134 of the stator 130 can receive the drive signal, which can be intermittently on or off to create the oscillatory field from the stator due to the drive signal and the magnetic field from the magnetic rotor 140. The magnetic rotor 140 rotates within the rotor gap 132 in response to the oscillatory field. The pump 150 operably connected to the magnetic rotor 140 moves the fluid from the reservoir 160 to the user 170 in response to the rotation of the magnetic rotor 140.

Those skilled in the art will appreciate that the pump 150 can be any pump operable to provide a well regulated, metered flow of the fluid to the user 170. The pump speed and thus the fluid delivery rate of the pump 150 can be controlled electronically by altering the signals between the electrical components of the personal injection device 100 or mechanically by gear ratios of gear chains between the rotor 140 and the pump 150, if any. In one example, the personal injection device 100 further includes a slide potentiometer operably connected to the control circuit 120 to adjust the drive signal sent to the stator 130.

The components of the personal injection device 100 can be enclosed within a casing for convenience of the user 170. The reservoir 160 can be placed inside or outside of the casing as desired for a particular application. In one embodiment, the reservoir 160 is prefilled and the personal injection device 100 discarded after a single use when the reservoir 160 is empty. The pump 150 can be attached to the user 170 with an infusion set or with a cannula projecting from the personal injection device 100.

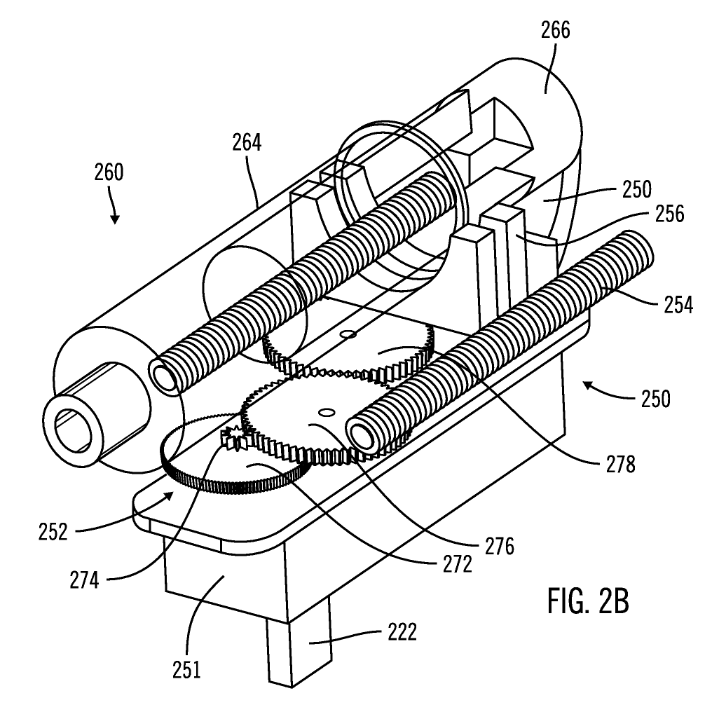

FIGS. 2A & 2B are perspective and top view diagrams, respectively, of a syringe pump for use with a personal injection device made in accordance with the invention. A syringe acts as the reservoir for the fluid to be delivered to the user.

The reservoir 260 is a syringe having a syringe body 264 and a syringe plunger 266 slideably disposed in the syringe body 264. The pump 250 includes a gear chain 252 operably connected to the magnetic rotor 240; screw drives 254 operably connected to the gear chain 252; a syringe body carriage 256 operable to removably receive the syringe body 264; and a syringe plunger carriage 258 operable to removably receive the syringe plunger 266. The screw drive 254 engages one of the syringe body carriage 256 and the syringe plunger carriage 258, so that the rotation of the magnetic rotor 240 moves the syringe body carriage 256 relative to the syringe plunger carriage 258 to advance the syringe plunger 266 in the syringe body 264. The relative motion increases pressure in the reservoir to drive fluid to the user.

Those skilled in the art will appreciate that the syringe plunger 266 and the syringe body 264 only need to be moved relative to each other to force the fluid from the syringe, i.e., either the syringe plunger 266 or the syringe body 264 can be held in a fixed position. In one embodiment as illustrated in FIGS. 2A & 2B, the screw drive 254 engages the syringe body carriage 256 and the syringe plunger carriage 258 is fixed relative to the magnetic rotor 240 and the pump body 251. In this embodiment, the gear chain 252 includes in turn a first gear 270 attached to the magnetic rotor 240, a second gear 272 affixed to a third gear 274, a fourth gear 276 which engages one of the screw drives 254, and a fifth gear 278 which engages the other of the screw drives 254. The syringe plunger carriage 258 is moved axially by the screw drives 254, which engage threaded portions of the syringe plunger carriage 258, to move the syringe plunger 264 and the syringe plunger carriage 258 is fixedly attached to the pump body 251 to hold the syringe plunger 266 in place. In another embodiment, the screw drives 254 engage the syringe plunger carriage 258 and the syringe body carriage 256 is fixed relative to the magnetic rotor 240 and the pump body 251. Also illustrated in FIG. 2A and disposed on the pump body 251 are the stator 230, the quartz oscillator 210, the control circuit 220, and the battery 280, which in this example is a button cell battery.

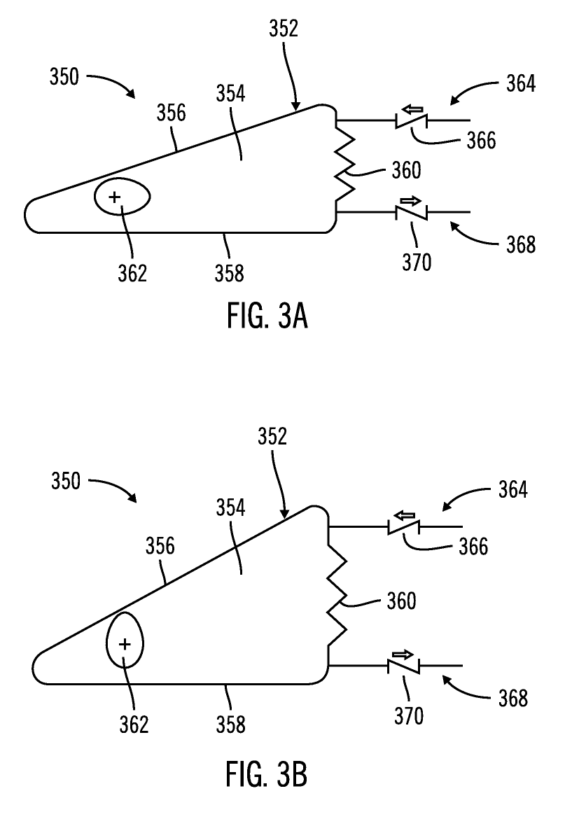

FIGS. 3A & 3B are schematic diagrams of a cam pump for use with a personal injection device made in accordance with the invention. FIG. 3A illustrates the pump with the volume of the plenum minimized after delivering fluid to the patient and FIG. 3B illustrates the pump with the volume of the plenum maximized after taking in fluid from the reservoir.

The pump 350 includes a pump wall 352 defining a plenum 354 having a volume, the pump wall 352 including a first wall 356, a second wall 358 hinged to the first wall 356, and a bellows wall 360 connected between the first wall 356 and the second wall 358, the bellows wall 360 urging the first wall 356 toward the second wall 358; a cam 362 operably connected to the magnetic rotor (not shown), the cam 362 being rotatably disposed in the plenum 354 between the first wall 356 and the second wall 358; a first fluid supply path 364 including an inlet check valve 366 in fluid communication between the reservoir (not shown) and the plenum 354; and a second fluid supply path 368 including an outlet check valve 370 in fluid communication between the plenum 354 and the user (not shown). The first wall 356 follows the cam 362 so that the volume of the plenum 354 is maximized when the cam 362 is in a first position as illustrated in FIG. 3B, and the volume of the plenum 354 is minimized when the cam 362 is in a second position as illustrated in FIG. 3A.

In operation, the cam 362 rotates with the first wall 356 following. As the first wall 356 moves away from the second wall 358, the volume of the plenum 354 increases, decreasing the pressure within the plenum 354 and drawing fluid from the reservoir (not shown) through the first fluid path 364 and inlet check valve 366. The outlet check valve 370 prevents backflow from the user (not shown) into the plenum 354. As the cam 362 rotates further, the first wall 356 moves toward the second wall 358 and the volume of the plenum 354 decreases, increasing the pressure within the plenum 354 to force fluid from the plenum 354 through the second fluid path 368 and the outlet check valve 370 to the user (not shown). The inlet check valve 366 prevents backflow from the plenum 354 into the reservoir (not shown). The bellows portion of the bellows wall 360 controls the force of the first wall 356 on the cam 362. Those skilled in the art will appreciate that the profile of the cam 362 can be selected as desired for particular pumping characteristics.

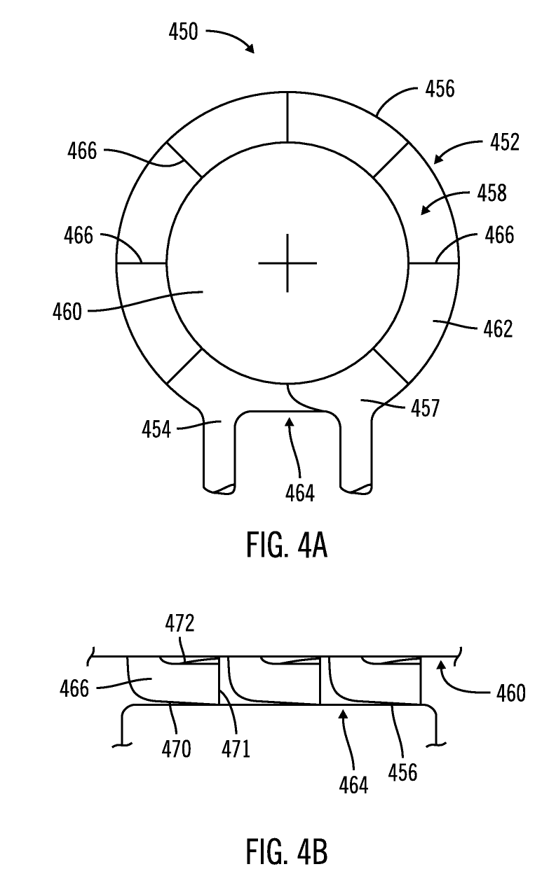

FIGS. 4A & 4B are schematic and detail diagrams, respectively, of a rotary pump for use with a personal injection device made in accordance with the invention.

Referring to FIG. 4A, the pump 450 includes a pump casing 452 having an inlet port 454 in fluid communication with the reservoir (not shown) and an outlet port 457 in fluid communication with the user (not shown), the pump casing 452 having a pump wall 456 defining a pump cavity 458; a pump rotor 460 operably connected to the magnetic rotor (not shown), the pump rotor 460 being rotatably disposed in the pump cavity 458, the pump rotor 460 and the pump wall 456 defining an annulus 462 with a narrowed portion 464 between the inlet port 454 and the outlet port 457; and flexible fingers 466 connected to the pump rotor 460, the flexible fingers 466 being disposed in the annulus 462 and in sealing contact with the pump wall 456. The flexible fingers 466 are sized and radially spaced to bend against the pump wall 456 in the narrowed portion 464 to draw the fluid into the inlet port 454 and to force the fluid out of the outlet port 457.

In operation, adjacent flexible fingers 466 trap a small volume of fluid at the inlet port 454 from the reservoir (not shown) and rotate the small volume around the annulus 462. When one of the flexible fingers 466 reaches the narrowed portion 464 of the annulus 462, the flexible finger bend backwards against the direction of rotation of the pump rotor 460, compressing the small volume between the bent flexible finger and the adjacent trailing flexible finger, increasing pressure at the outlet port 457 to drive the fluid to the user (not shown). Those skilled in the art will appreciate that size and radial spacing of the flexible fingers 466 can be selected to achieve desired pumping characteristics.

Referring to FIG. 4B, in one embodiment the bent flexible fingers 466 fill the narrowed portion 464. The flexible fingers 466 are sized and radially spaced so that, when bent in the narrowed portion 464, the leading side 470 of the flexible finger is in contact with the pump wall 456, the trailing side 472 of the flexible finger is in contact with the pump rotor 460, and the tip 471 of the flexible finger is adjacent the next adjacent trailing flexible finger. As defined herein, the bent flexible fingers fill the narrowed portion when the bent flexible fingers occupy more than 90 percent of the volume of the narrowed portion of the annulus.

It is important to note that FIGS. 1-4 illustrate specific applications and embodiments of the invention, and are not intended to limit the scope of the present disclosure or claims to that which is presented therein. Upon reading the specification and reviewing the drawings hereof, it will become immediately obvious to those skilled in the art that myriad other embodiments of the invention are possible, and that such embodiments are contemplated and fall within the scope of the presently claimed invention.

While the embodiments of the invention disclosed herein are presently considered to be preferred, various changes and modifications can be made without departing from the spirit and scope of the invention. The scope of the invention is indicated in the appended claims, and all changes that come within the meaning and range of equivalents are intended to be embraced therein.

1. A personal injection device wearable by a user to deliver fluid from a reservoir to the user, the personal injection device comprising:
a quartz oscillator operable to generate a constant frequency signal;
a control circuit operably connected to the quartz oscillator, the control circuit being operable to generate a drive signal in response to the constant frequency signal;
a stator operably connected to the control circuit and defining a rotor gap, the stator being operable to generate an oscillatory field in the rotor gap in response to the drive signal;
a magnetic rotor disposed in the rotor gap, the magnetic rotor being operable to rotate in response to the oscillatory field; and
a pump operably connected to the magnetic rotor, the pump being operable to move the fluid from the reservoir to the user in response to the rotation of the magnetic rotor.

2. The personal injection device of claim 1 further comprising a slide potentiometer operably connected to the control circuit to adjust the drive signal.

3. The personal injection device of claim 1 wherein the reservoir comprises a syringe having a syringe body and a syringe plunger slideably disposed in the syringe body, the pump comprising:
a gear chain operably connected to the magnetic rotor;
a screw drive operably connected to the gear chain;
a syringe body carriage operable to removably receive the syringe body; and
a syringe plunger carriage operable to removably receive the syringe plunger;
wherein the screw drive engages one of the syringe body carriage and the syringe plunger carriage, the rotation of the magnetic rotor moving the syringe body carriage relative to the syringe plunger carriage to advance the syringe plunger in the syringe body.

4. The personal injection device of claim 3 wherein the screw drive engages the syringe body carriage and the syringe plunger carriage is fixed relative to the magnetic rotor.

5. The personal injection device of claim 3 wherein the screw drive engages the syringe plunger carriage and the syringe body carriage is fixed relative to the magnetic rotor.

6. The personal injection device of claim 1 wherein the pump comprises:
a pump wall defining a plenum having a volume, the pump wall including a first wall, a second wall hinged to the first wall, and a bellows wall connected between the first wall and the second wall, the bellows wall urging the first wall toward the second wall;
a cam operably connected to the magnetic rotor, the cam being rotatably disposed in the plenum between the first wall and the second wall;
a first fluid supply path including an inlet check valve in fluid communication between the reservoir and the plenum; and
a second fluid supply path including an outlet check valve in fluid communication between the plenum and the user;
wherein the first wall follows the cam, the volume of the plenum is maximized when the cam is in a first position, and the volume of the plenum is minimized when the cam is in a second position.

7. The personal injection device of claim 1 wherein the pump comprises:
a pump casing having an inlet port in fluid communication with the reservoir and an outlet port in fluid communication with the user, the pump casing having a pump wall defining a pump cavity;
a pump rotor operably connected to the magnetic rotor, the pump rotor being rotatably disposed in the pump cavity, the pump rotor and the pump wall defining an annulus with a narrowed portion between the inlet port and the outlet port; and
flexible fingers connected to the pump rotor, the flexible fingers being disposed in the annulus and in contact with the pump wall;
wherein the flexible fingers are sized and radially spaced to bend against the pump wall in the narrowed portion to draw the fluid into the inlet port and to force the fluid out of the outlet port.

8. The personal injection device of claim 1 wherein the bent flexible fingers fill the narrowed portion.

9. A personal injection device wearable by a user to deliver fluid from a reservoir to the user, the personal injection device comprising:
a quartz oscillator operable to generate a constant frequency signal;
a control circuit operably connected to the quartz oscillator, the control circuit being operable to generate a drive signal in response to the constant frequency signal;
a stator operably connected to the control circuit and defining a rotor gap, the stator being operable to generate an oscillatory field in the rotor gap in response to the drive signal;
a magnetic rotor disposed in the rotor gap, the magnetic rotor being operable to rotate in response to the oscillatory field; and
a pump operably connected to the magnetic rotor, the pump being operable to move the fluid from the reservoir to the user in response to the rotation of the magnetic rotor, the pump comprising:
a pump wall defining a plenum having a volume, the pump wall including a first wall, a second wall hinged to the first wall, and a bellows wall connected between the first wall and the second wall, the bellows wall urging the first wall toward the second wall;
a cam operably connected to the magnetic rotor, the cam being rotatably disposed in the plenum between the first wall and the second wall;
a first fluid supply path including an inlet check valve in fluid communication between the reservoir and the plenum; and
a second fluid supply path including an outlet check valve in fluid communication between the plenum and the user;
wherein the first wall follows the cam, the volume of the plenum is maximized when the cam is in a first position, and the volume of the plenum is minimized when the cam is in a second position.

10. The personal injection device of claim 9 wherein the inlet check valve is operable to allow flow to the plenum as the first wall moves away from the second wall and to block flow from the plenum as the first wall moves toward the second wall.

11. The personal injection device of claim 9 wherein the outlet check valve is operable to block flow to the plenum as the first wall moves away from the second wall and to allow flow from the plenum as the first wall moves toward the second wall.

12. The personal injection device of claim 9 wherein the bellows wall includes a bellows portion operable to control force of the first wall on the cam.

13. The personal injection device of claim 9 further comprising a slide potentiometer operably connected to the control circuit to adjust the drive signal.

14. A personal injection device wearable by a user to deliver fluid from a reservoir to the user, the personal injection device comprising:
a quartz oscillator operable to generate a constant frequency signal;
a control circuit operably connected to the quartz oscillator, the control circuit being operable to generate a drive signal in response to the constant frequency signal;
a stator operably connected to the control circuit and defining a rotor gap, the stator being operable to generate an oscillatory field in the rotor gap in response to the drive signal;
a magnetic rotor disposed in the rotor gap, the magnetic rotor being operable to rotate in response to the oscillatory field; and
a pump operably connected to the magnetic rotor, the pump being operable to move the fluid from the reservoir to the user in response to the rotation of the magnetic rotor, the pump comprising:
a pump casing having an inlet port in fluid communication with the reservoir and an outlet port in fluid communication with the user, the pump casing having a pump wall defining a pump cavity;
a pump rotor operably connected to the magnetic rotor, the pump rotor being rotatably disposed in the pump cavity, the pump rotor and the pump wall defining an annulus with a narrowed portion between the inlet port and the outlet port; and
flexible fingers connected to the pump rotor, the flexible fingers being disposed in the annulus and in contact with the pump wall;
wherein the flexible fingers are sized and radially spaced to bend against the pump wall in the narrowed portion to draw the fluid into the inlet port and to force the fluid out of the outlet port.

15. The personal injection device of claim 14 wherein the bent flexible fingers fill the narrowed portion.

16. The personal injection device of claim 14 further comprising a slide potentiometer operably connected to the control circuit to adjust the drive signal.
